(19) United States
(12) Patent Application Publication
(10) Pub. No.: US 2019/0039087 A1
(43) Pub. Date: Feb. 7, 2019

(54) POINT-OF-USE FLUID QUALITY MEASUREMENT

(71) Applicant: Gray Matter Systems LLC, Warrendale, PA (US)

(72) Inventors: Alan Hinchman, Warrendale, PA (US); Thomas L. Kuczynski, Washington, DC (US)

(21) Appl. No.: 16/054,310

(22) Filed: Aug. 3, 2018

Related U.S. Application Data
(60) Provisional application No. 62/541,745, filed on Aug. 6, 2017.
(60) Provisional application No. 62/541,746, filed on Aug. 6, 2017.

(51) Int. Cl. B05B 17/08; G01N 33/18; G01N 27/416; B05B 12/00; B05B 12/08

(52) U.S. Cl. CPC B05B 17/08; G01N 33/18; B05B 12/08; G01N 27/4168; B05B 12/004; G01N 27/4167

(57) ABSTRACT

One embodiment provides a point-of-use fluid delivery system, including: a vessel containing a fluid; at least one sensor capturing data related to a first measurement comprising a pH measurement; at least one other sensor capturing data related to a second measurement selected from the group consisting of: an ORP measurement and a conductivity measurement of the fluid; a processor; and a memory device operatively coupled to the processor and that stores instructions executable by the processor to transmit the data related to the pH and/or temperature measurement of the fluid and the data related to the conductivity and/or ORP measurement of the fluid to a remote system. Other aspects are described and claimed.

### CROSS REFERENCE TO RELATED APPLICATIONS

This application claims priority to Provisional Application No. 62/541,746, filed Aug. 6, 2017, and entitled “POINT-OF-USE FLUID QUALITY MEASUREMENT,” and to Provisional Application No. 62/541,745, filed Aug. 6, 2017, and entitled “DETERMINATION OF FLUID QUALITY USING pH AND RESISTIVITY,” the contents of which are incorporated by reference as if set forth in their entirety.

### BACKGROUND

Determining the quality of drinking water is a critical function. If drinking water has too many biological or organic materials it can cause health concerns and problems for a population. Similarly, if the drinking water contains too many metals or specific types of metal, the drinking water can pose a health hazard. Determining the quality of water is normally done using two methods. First with expensive in-line water quality testing equipment that can detect trace amounts of organics and/or metals. Additionally, this equipment can determine how much of the organics or metals are present in the water. The second method includes using samples from a location and then testing those samples at a water quality laboratory. This method is labor intensive and can result in false readings due to poor sampling procedures, loss of sample custody, contamination, and numerous other reasons. The cost and inconsistency of the manual sample method the preference is to use in-line water quality instruments.

Due to the cost of the equipment, water quality instrument based testing is usually done at a water treatment facility or water storage location. Thus, a water utility can ensure that the water leaving the facility is of sufficient quality to prevent health hazards. However, this testing does not take into account any organics or metals that are introduced to the water from the time the water leaves the facility to the time that it reaches the end consumer, for example, at a tap in a home, in a business, at a drinking fountain, and the like. For example, if the pipes that transport the water from the facility to the point-of-use location are corroded, the corrosion may be transferred to the water as it travels through the pipes. This transference would not be accounted for in the water quality testing completed at the facility. Thus, the water that a person is drinking may be of poor quality and included biologics or heavy metals that can cause serious health concerns.

### BRIEF SUMMARY

In summary, one aspect provides a point-of-use fluid delivery system, comprising: a vessel containing a fluid; at least one sensor capturing data related to a first measurement comprising a pH measurement; at least one other sensor capturing data related to a second measurement selected from the group consisting of: an ORP measurement and a conductivity measurement of the fluid; a processor; and a memory device operatively coupled to the processor and that stores instructions executable by the processor to transmit the data related to the pH and/or temperature measurement of the fluid and the data related to the conductivity and/or ORP measurement of the fluid to a remote system.

Another aspect provides a system, comprising: a processor; a memory device operatively coupled to the processor, wherein the memory device comprises instructions executable by the processor to: capture, using at least one sensor of a fluid delivery system, data related to a first measurement comprising a pH measurement of a fluid sample; capture, using at least one other sensor of the fluid delivery system, data related to a second measurement selected from the group consisting of: an ORP measurement and a conductivity measurement of the fluid sample; and transmit the first measurement data and the second measurement data to a remote system for identifying a quality of the fluid sample determined by correlating the first measurement data and the second measurement data to quality information.

A further aspect provides a water fountain system, comprising: tubing containing water; a water sample chamber connected to the tubing, wherein the water sample chamber draws water into the water sample chamber; at least one sensor, connected to the water sample chamber, capturing data related to a first measurement comprising a pH measurement of the fluid; at least one other sensor, connected to the water sample chamber, capturing data related to a second measurement selected from the group consisting of: an ORP measurement and a conductivity measurement of the fluid; a data transmission device; a processor; and a memory device operatively coupled to the processor and that stores instructions executable by the processor to transmit, using the data transmission device, the data related to the first measurement of the fluid and the data related to the second measurement of the fluid to a remote system.

A further aspect provides for the communication and collection of individual sensor pairs at the point of use devices to a centralized time series database and analytics software platform. The time series database may cleanse and normalize the data so that the analytics platform can compare the difference. For example, water from the same source with same treatment should have similar chemistry. Thus, comparing different measurements may indicate water quality issue in the delivery system, water age, and possible tampering. Centralized comparison data can be combined with laboratory testing and/or inline water quality monitoring in the water plants and distribution system.

A further aspect is the individual data storage and correlation of the individual sensors to determine quality of the data received and health of the probe. Life-long comparison based on initial probe calibration and readings versus long term tread can determine failure of probes. Where similar measurement devices are used in similar fluid, a correction could be made from the correlation the life-long data trends and related probes to increase useful life by correcting the data.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example method of determining the quality of a fluid using pH, which may include temperature correction, and conductivity/ORP measurements.

FIG. 4 illustrates an example point-of-use quality measurement system.

FIG. 5 illustrates an example of the communication of a single device to a database.

FIG. 6 illustrates an example of the networking of multiple devices.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Water quality collection and testing uses expensive instrumentation that is usually done by the water company or done in a lab. Accordingly, the current method is to test the water at the water treatment facility as the water is leaving the facility. However, this does not necessarily mean that by the time the water reaches its destination (e.g., hospital, school, a person's home, a business, etc.) that it is still of the same quality. Rather, if the pipes and other transportation vessels are of bad quality (e.g., rusting, corroding, contain bacteria, etc.) the water will pick up these contaminants as it travels through the transportation vessels. Thus, the water may actually be of very poor quality by the time it reaches its destination for drinking, cooking, or other use by a person. The bad water quality could cause many health concerns or issues. However, testing the water quality at each destination point is very expensive due to the instrumentation that would be needed to perform these types of tests. Accordingly, testing water at each destination point is cost prohibitive and unlikely to be done.

One solution has been to add some water quality testing instrumentation to periodic locations on the fluid transportation route. For example, some water quality measurement instrumentation may be added to piping or junctions within the fluid transportation system. As another example, water quality measurement instrumentation may be added to fluid storage facilities or other storage locations along the fluid transportation system. However, the addition of this instrumentation still does not provide for an accurate indication or identification of the quality of the water once it has reached the point-of-use destination.

In 1974, Congress passed the Safe Drinking Water Act. This law requires the EPA to determine the level of residual disinfectants in drinking water at which no adverse health effects are likely to occur. These non-enforceable health goals, based solely on possible health risks and exposure over a lifetime, with an adequate margin of safety, are called maximum residual disinfectant level goals (MRDLG). Contaminants are any physical, chemical, biological or radiological substances or matter in water. The EPA sets MRDLGs based on the best available science to prevent potential health problems. Based on the MRDLG, the EPA has set enforceable regulations for disinfectants, called a maximum residual disinfectant level (MRDL), at the following levels:

| Disinfectant | MRDLG | MRDL |
|---|---|---|
| Chloramine | 4 milligrams per | 4.0 mg/L or 4 |
| | liter (mg/L) or 4 | ppm as an |
| | parts per million | annual average |
| | (ppm) | |
| Chlorine | 4 mg/L or 4 | 4.0 mg/L or 4 |
| | ppm | ppm as an |
| | | annual average |
| Chlorine | 0.8 mg/L or 800 | 0.8 mg/L or 800 |
| Dioxide | parts per billion | ppb |
| | (ppb) | |

MRDLs are set as close to the health goals as possible, considering cost, benefits and the ability of public water systems to detect and remove contaminants using suitable treatment technologies. In this case, the MRDL equals the MRDLG, because analytical methods or treatment technology do not pose any limitations. States may set more stringent drinking water MRDLGs and MRDLs for disinfectants than the EPA.

Some water quality measurements are measured by collecting and measuring a total amount of chlorine in the water. Chlorine is the disinfectant that is added to water to remove biologics and/or organics. The more biologics that are in the water the more chlorine that has to be added to remove the biologics. The US EPA manages water quality inside a window between MRDLGs and MRDLs. Accordingly, a measurement of volume of chlorine in the water outside this window provides an indication that the water is of poor quality. However, in order to accurately measure the amount of chlorine in the water, different chemicals or reagents have to be added to the measurement sample. The addition of these chemicals or reagents increases the cost associated with these measurements because not only may the chemicals and reagents be expensive, but they also have to be periodically added to the instrumentation so that the measurements can be taken. Thus, the instrumentation also requires periodic maintenance to at least add the necessary chemicals or reagents thereby adding to the cost of the instrumentation.

Accordingly, an embodiment provides a system and method for determining the quality of water by calculating the water quality rather than taking a direct measurement of the water quality. For example, rather than taking a direct measurement of the chlorine contained within the water, an embodiment measures the pH, which may include temperature correction, and the conductivity and/or oxidation reduction potential (ORP) of the water. However, one skilled in the art of water quality will understand the disinfection method used and reason for usage. This knowledge will determine the selection of the measures used. Typical measurements would include pH and conductivity or pH and ORP. The ORP measurement may be commercially known as oxidation reduction potential (ORP) measurement. The conductivity measurement may be commercially known as conductance or conductivity measurement. Conductivity is the measure of water's ability to transfer electrical current. This transfer is directly related to presence of dissolved solids.

Using the pH measurement, which may include temperature correction, and conductivity and/or ORP measurements, an embodiment may access an information store including information related to quality levels of fluids. For example, in one embodiment the information store may include a look up table which provides a correlation between the temperature corrected pH and conductivity and/or ORP measurements and a water quality level as compared to a threshold range. In some situations, the fluid measured may require conductivity and ORP without the use of pH. In this case the embodiment the information store may include a look up table or formula which provides a correlation between the ORP and conductivity measurements and a water quality level as compared to a threshold range. For ease of readability the discussion herein will refer to water and water quality. However, it should be understood by one skilled in the art that the systems and methods as described herein could apply to other fluids, for example, oil, water other than drinking water, and the like.

An embodiment may then determine the quality of the fluid by calculating or correlating the pH, which may include temperature correction, and conductivity/ORP measurements to the quality information included in the information store. Determining the quality may include identifying if the quality has been indicated as outside a predetermined threshold or window which indicates that the water may be of poor quality. In response to determining that the water is of poor quality, an embodiment may perform a function. For example, if the water is of poor quality an embodiment may provide instructions to shut-off or otherwise disable the point-of-use device. As another example, an embodiment may provide an alert to one or more users that the water is of poor quality. An embodiment may also correlate information received from multiple users or entities to determine a location of contamination.

One embodiment may be implemented in a water delivery system, for example, a water fountain, home water delivery system (e.g., sink, water heater, water filtration system, etc.), business water delivery system, municipal water delivery system, and the like. For ease of readability, the example of a water fountain will be used here throughout. However, it should be understood that this is a non-limiting example and is only used for ease of readability and clarity. The water fountain may include vessels (e.g., tubing, piping, etc.) for transporting the water from the inlet (e.g., building piping) to the outlet (e.g., water bubbler). The water fountain may include the traditional water fountain components. Additionally, the water fountain may include one or more sensors for capturing a pH measurement which may include a temperature correction of the water and one or more sensors for capturing conductivity/ORP measurements of the water.

In the case of the installation of multiple monitoring devices for a single source of water, such measurement groups may be compared and provide collaboration or difference of the measurements. Using comparative analytics, a resulting action would be taken of data correction or the determination of a water quality issue. On the determination of a water quality issue an alarm via software display or messaging (SMS or email) may be sent. Additionally or alternatively, a local alarm light or display may also illuminate. Under predefined software based rules the system may also stop the water flow to multiple impacted units.

The water fountain may also include components for either processing the measurement data or for transmitting the measurement data to a remote system for processing. The processing may include determining the quality of the water, as explained in more detail herein. The water fountain may also include components that can be controlled using instructions for disabling the water fountain, for example, a shut-off mechanism. This shut-off mechanism may be activated in response to receiving instructions or an indication that the water quality is poor. Accordingly, an embodiment provides a system and method for ensuring that the water quality received at the point-of-use location is of acceptable quality.

The illustrated example embodiments will be best understood by reference to the figures. The following description is intended only by way of example, and simply illustrates certain example embodiments.

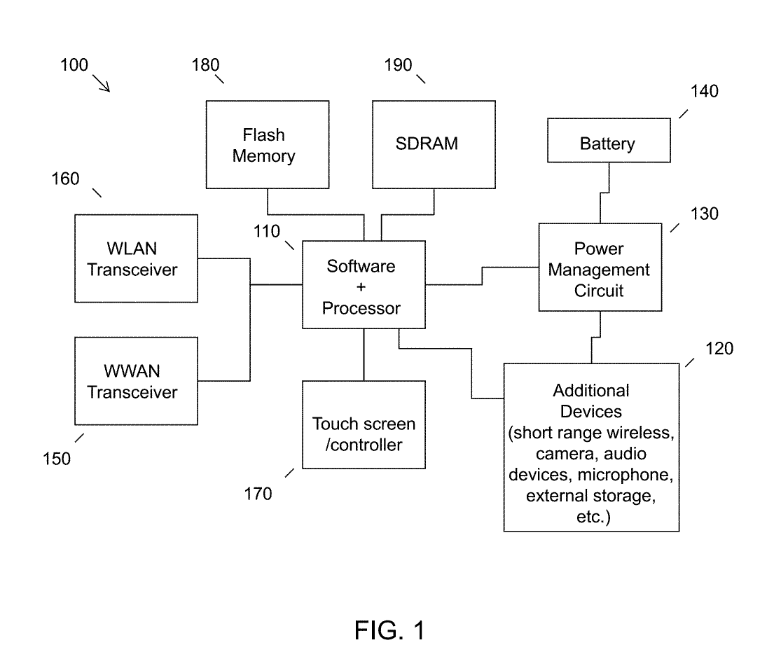

While various other circuits, circuitry or components may be utilized in information handling devices, with regard to smart phone and/or tablet circuitry 100, an example illustrated in FIG. 1 includes a system on a chip design found for example in tablet or other mobile computing platforms. Software and processor(s) are combined in a single chip 110. Processors comprise internal arithmetic units, registers, cache memory, busses, I/O ports, etc., as is well known in the art. Internal busses and the like depend on different vendors, but essentially all the peripheral devices (120) may attach to a single chip 110. The circuitry 100 combines the processor, memory control, and I/O controller hub all into a single chip 110. Also, systems 100 of this type do not typically use SATA or PCI or LPC. Common interfaces, for example, include SDIO and I2C.

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply BIOS like functionality and DRAM memory.

System 100 typically includes one or more of a WWAN transceiver 150 and a WLAN transceiver 160 for connecting to various networks, such as telecommunications networks and wireless Internet devices, e.g., access points. Additionally, devices 120 are commonly included, e.g., an image sensor such as a camera. System 100 often includes a touch screen 170 for data input and display/rendering. System 100 also typically includes various memory devices, for example flash memory 180 and SDRAM 190.

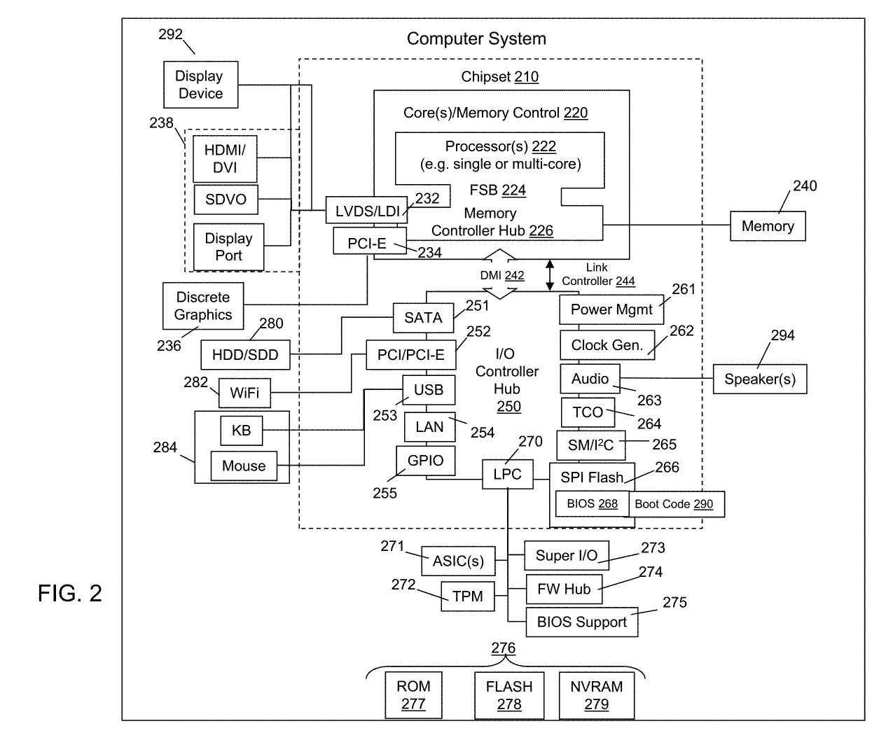

FIG. 2 depicts a block diagram of another example of information handling device circuits, circuitry or components. The example depicted in FIG. 2 may correspond to computing systems such as the THINKPAD series of personal computers sold by Lenovo (US) Inc. of Morrisville, N.C., or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer (for example, INTEL, AMD, ARM, etc.). INTEL is a registered trademark of Intel Corporation in the United States and other countries. AMD is a registered trademark of Advanced Micro Devices, Inc. in the United States and other countries. ARM is an unregistered trademark of ARM Holdings plc in the United States and other countries. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of RAM that may be referred to as “system memory” or “memory”). The memory controller hub 226 further includes a low voltage differential signaling (LVDS) interface 232 for a display device 292 (for example, a CRT, a flat panel, touch screen, etc.). A block 238 includes some technologies that may be supported via the LVDS interface 232 (for example, serial digital video, HDMI/DVI, display port). The memory controller hub 226 also includes a PCI-express interface (PCI-E) 234 that may support discrete graphics 236.

In FIG. 2, the I/O hub controller 250 includes a SATA interface 251 (for example, for HDDs, SDDs, etc., 280), a PCI-E interface 252 (for example, for wireless connections 282), a USB interface 253 (for example, for devices 284 such as a digitizer, keyboard, mice, cameras, phones, microphones, storage, other connected devices, etc.), a network interface 254 (for example, LAN), a GPIO interface 255, a LPC interface 270 (for ASICs 271, a TPM 272, a super I/O 273, a firmware hub 274, BIOS support 275 as well as various types of memory 276 such as ROM 277, Flash 278, and NVRAM 279), a power management interface 261, a clock generator interface 262, an audio interface 263 (for example, for speakers 294), a TCO interface 264, a system management bus interface 265, and SPI Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Information handling device circuitry, as for example outlined in FIG. 1 or FIG. 2, may be used in devices such as tablets, smart phones, personal computer devices generally, and/or electronic devices which may be used to analyze captured or received measurement information to determine a quality of a fluid. Additionally, the devices may be included within or coupled to a fluid delivery system to capture measurements. For example, the circuitry outlined in FIG. 1 may be implemented in a tablet or smart phone embodiment, whereas the circuitry outlined in FIG. 2 may be implemented in a personal computer embodiment.

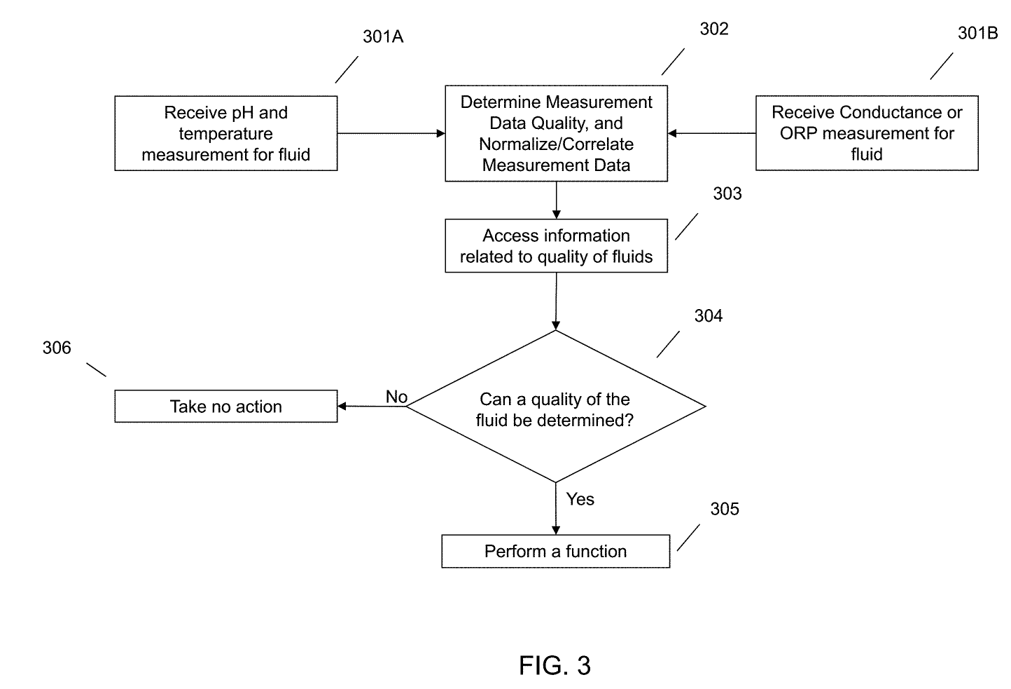

Referring now to FIG. 3, an embodiment may determine a quality of a fluid using surrogate measurements to calculate the quality rather than obtaining a direct measurement of the water quality. At 301A an embodiment may receive a pH measurement, which may include temperature correction, of a fluid from one or more sensors. The fluid may be contained within one or more vessels, for example, piping, tubing, chamber, or the like. The one or more sensors may be included within a device or system that is in the transportation network of the fluid. For example, if the system includes a point-of-use water delivery system the sensors may be included in the point-of-use water delivery system.

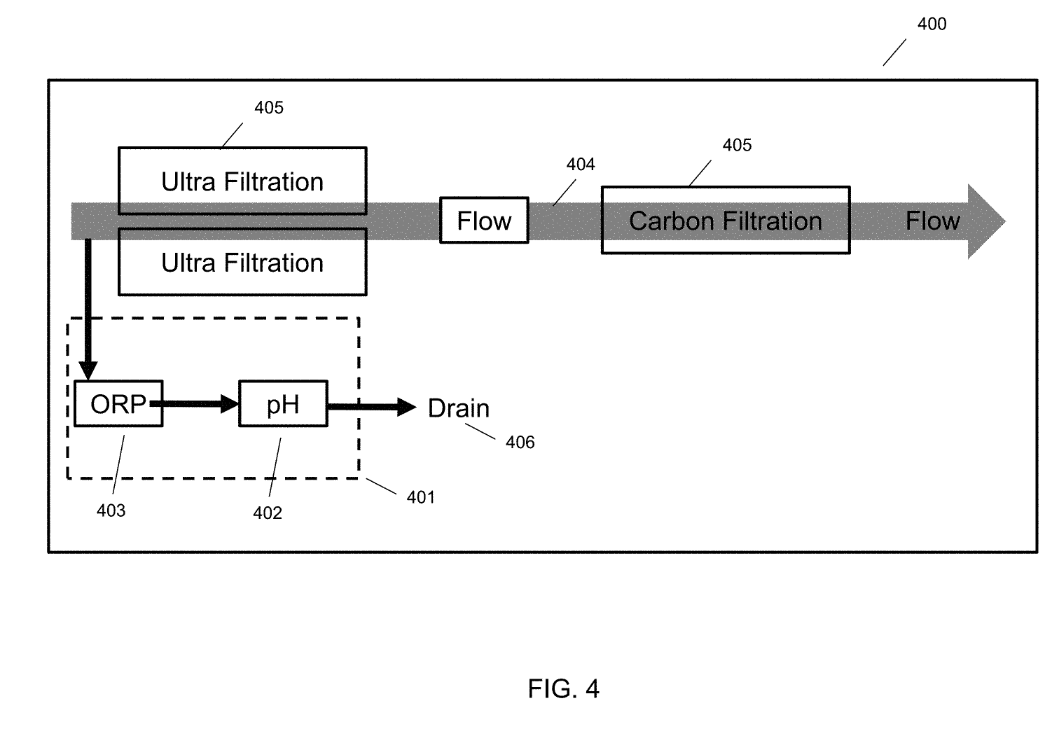

The example that will be used here throughout will be that of a water fountain, for example, as shown in FIG. 4. The water fountain 400 may include the parts and components of a traditional water fountain, for example, water filtration 405, tubing or other fluid vessel 404, and the like. In addition, the water fountain 400 may include one or more sensors for capturing pH measurement data, which may be corrected based on the temperature of the fluid, 402 and/or one or more sensors for capturing conductivity or oxidation-reduction potential (ORP) measurement data 403. The one or more sensors may be in-line with the fluid vessel 404. For example, if the fluid vessel includes tubing, the one or more sensors may be contained within the tubing, within a junction in the tubing, or the like. In one embodiment the fluid may be drawn into a fluid sample chamber, for example, as shown at 401 in FIG. 4. The sensors may be integral to, operatively coupled to, or otherwise at or connected to the fluid sample chamber. In the case that the system includes a fluid sample chamber, the fluid sample chamber may include a flow to a drain 406 or other disposal area to discard the sample after capturing the desired measurements.

The pH sensors, which may also include temperature sensors for temperature correction of the pH measurement, may measure the pH, which may be corrected based upon the temperature, level of the fluid. Such a measurement may provide an indication of level of biological activity within the water. In other words, the pH, which may include temperature correction, measurement may give an indication of organics or biologics (e.g., bacteria, viruses, etc.) within the water. Biological activity lowers the pH with temperature correction of water. Therefore, a lower than expected value for the pH with temperature correction level may indicate the presence of biological activity. A higher than expected value for the pH, which may include temperature correction, level may be an indicator of really poor water quality.

At 301B an embodiment may receive a conductivity or oxidation-reduction potential (ORP) measurement of the fluid from one or more sensors. These sensors may be located similarly to the pH and/or temperature sensors. Thus, the description of the locations of these sensors will not be reiterated here. However, it should be understood that the description of the pH and/or temperature sensors is applicable to the conductivity/ORP measurement sensors. Additionally, it should be understood that the pH and/or temperature sensors and the conductivity/ORP measurement sensors do not have to be located in the same location for every device. For example, in one device the conductivity/ORP sensor may be located in-line with the tubing and the pH and/or temperature sensor may be located within the fluid sample chamber.

The conductivity measurement may provide an indication of the current potential or conductivity of the water, which may provide an indication of metal within the water. As an example, pure water has a particular resistance value. An increase in the metallic content of the water may increase the conductivity of the water. Therefore, the resistance value may decrease indicating a conductivity of the water. Conversely, a higher conductivity value may indicate the inclusion of a substance having a lower conductivity than pure water. Accordingly, the conductivity measurement may provide an indication of metals or other particles in the water that change the conductivity or conductivity of the water. The conductivity measurement sensor may include a sensor for measuring the oxidation-reduction potential. As explained above, the oxidation-reduction potential value may provide an indication of metal in the water, for example, as may be caused by corrosion of a pipe that the water has traveled through.

ORP is measured to determine the oxidizing or reducing potential of a water sample. It indicates possible contamination of drinking water. One skilled in the art may understand, know, or find it valuable to determine the component of the sample that is contributing to the ORP value. In other words, one component of the sample is primarily responsible for the observed ORP value. For example, excess chlorine in wastewater effluent will result in a large positive ORP value and the presence of hydrogen sulfide will result in a large negative ORP value. ORP is determined by measuring the potential of a chemically-inert (platinum) electrode which is immersed in the solution. The sensing electrode potential is read relative to the reference electrode of the pH probe and the value is presented in millivolts (mV). The determination of ORP is generally significant in water which contains a relatively high concentration of a redox-active species, e.g., the salts of many metals (Fe2+, Fe3+) and strong oxidizing (chlorine) and reducing (sulfite ion) agents. Thus, ORP can sometimes be utilized to track the metallic pollution in groundwater or surface water or to determine the chlorine content of wastewater effluent. However, ORP is a nonspecific measurement, i.e., the measured potential is reflective of a combination of the effects of all the dissolved species in the medium.

At 302, receiving the pH measurement, which may include temperature correction, and/or the conductivity/ORP measurement may include receiving or otherwise obtaining the measurement. For example, in one embodiment the measurements or data derived from the measurements may be transmitted to a remote storage location (e.g., network data storage, cloud storage, etc.). The measurements may also be transmitted to a remote processing/analysis system (e.g., cloud processing system, remote processing system, third party processing system, etc.). As another example, in one embodiment the measurements may be accessed by an embodiment. For example, a remote processing/analysis system may access the device including the sensors and access the measurement data from the device. As another example, the measurement data may be sent to a data storage location that is then accessed by the processing system. In other words, receiving the measurement data may include receiving, obtaining, accessing, or otherwise capturing the measurement data from the device housing the sensors, from a third-party data storage system, from a processing system, or the like. Accordingly, the point-of-use fluid delivery system may include a data transmission device that can transmit and receive data from the system to the processing/analysis system. The data transmission device may include a wired device (e.g., connected to a system using a wire, etc.), a wireless device (e.g., connected to a system using wireless signals, etc.), or a combination thereof.

Both the pH measurements, which may include temperature correction, and conductivity/ORP measurements may be taken in response to an instruction to take the measurements. In one embodiment, a user or remote system may provide an input that causes the measurements to be taken. For example, a user may press a measurement button located on the fountain, a user may send a signal from a remote location to take a measurement, a remote system may need measurements for analysis and may provide an instruction to take measurements, or the like. In one embodiment, the measurements may be taken at predetermined intervals. For example, the measurements may be taken every fifteen minutes. The predetermined intervals may be a default interval or may set by a user or remote system based upon different factors. For example, a user may identify a possible problem with the water and may therefore set the system to take measurements at shorter intervals than normally taken.

At 303 an embodiment may access information related to quality of fluids. This may assist in analyzing the pH measurement, which may include temperature correction, and conductivity/ORP measurements. In one embodiment the information related to quality of fluids may include a look up table that identifies correlations between pH measurements, which may include temperature correction, and conductivity/ORP measurements and water quality. An embodiment may then use the correlation to calculate the water quality. In one embodiment the information related to quality of fluids may include a computer program that uses the measurement values to estimate a water quality. In an embodiment the water quality may not be a specific value. Rather, the information may identify a sample outside the operating window indicates poor water quality. As an example, using the pH, which may include temperature correction, an embodiment may determine that pH, which may include temperature correction, above a certain value may indicate poor water quality. However, the system may not identify a specific value associated with the water quality.

In one embodiment a temperature value of the water may also be used to determine the water quality. The temperature value may be captured using another sensor on the water fountain system. Alternatively, the temperature value may be a static value that is based upon a common or average temperature value. For example, a user may set the temperature value used for analysis based upon the common temperature of the water at an outlet of the water transportation system. The information related to the quality of fluids may include the temperature. Accordingly, the system may use the temperature value, the pH measurement (which may be corrected based upon the temperature), and the conductivity/ORP measurement and correlate all of these values to a water quality, as explained in more detail below.

In one embodiment the information may include information related to a level of chlorine indicating the water quality. Accordingly, in one embodiment the measurement data is used to calculate an amount of chlorine in the fluid sample. For example, rather than directly measuring the amount of chlorine in the water, an embodiment may make a correlation that an X value for a pH measurement, which may be corrected for a temperature value, and a Y value for a conductivity/ORP measurement at a Z temperature indicates a particular level of chlorine. This level of chlorine may then be used to identify a quality of the water.

At 304 an embodiment may determine whether a quality of the fluid within the fluid delivery system can be determined. Determining the quality of the fluid may include correlating the received pH measurement, which may include temperature correction, and the received conductivity/ORP measurement to the accessed information. For example, in the example of a look up table, an embodiment may use the values of the pH with temperature correction measurement and conductivity/ORP measurement to determine the correlation to a quality within the look up table. An embodiment may additionally use the temperature value, either as measured or as provided by a user, in the correlation. In one embodiment, the pH measurement (which may be corrected based upon the temperature value), conductivity/ORP measurement, and/or the temperature value may be mapped to a chlorine level or an amount of chlorine. The chlorine level may then be correlated to a quality of the water.

As stated above, determining a quality of the fluid may not include determining a specific quality of the fluid. For example, the systems and methods as described herein may not provide a specific indication of a specific level of biologics, metals, or other contaminants. Additionally, the systems and methods as described herein may not identify the type of biologics, metals, or other contaminants. In other words, using the techniques as described herein the system may be unable to identify specific contaminants or specific levels of contaminants. Rather, the systems and methods as described herein may only provide an indication of a water quality level as compared to a threshold or range. Accordingly, in one embodiment determining the quality may include determining whether the quality is outside an expected or acceptable range or threshold value. This determination may be based upon the accessed information which may give an indication that a pH value, which may be corrected for a temperature, and/or conductivity/ORP value outside a particular range indicates a poor quality, while a pH value, which may be corrected for a temperature, and/or conductivity/ORP value within a particular range indicates an acceptable quality.

If a quality of the fluid cannot be determined at 304, an embodiment may take no action at 306. Alternatively, if the system determines that the quality of the fluid is within acceptable ranges, the system may take no further action. If, however, the quality of the fluid can be determined at 304, an embodiment may perform a function at 305. Performance of a function may not only be associated with poor water quality, but may also be completed if the quality is determined to be acceptable or good. However, the specific function may be different or modified depending on the results of the determination. For example, if an embodiment determines that the quality is acceptable or good, an embodiment may provide a notification to a user of the results of the determination. Whereas, if an embodiment determines that the quality is poor, an embodiment may provide instructions to shut-off the device.

In one embodiment performance of a function may include merely providing an indication of the quality. For example, an embodiment may provide an indication of whether the quality of the fluid is bad, acceptable, good, poor, unacceptable, or the like. Additionally, the system may merely provide a visual indication of the quality of the fluid, for example, a green indicator for good and a bad indicator for bad. As another example, the system may provide a check mark for good quality and an X for bad quality. As should be understood by one skilled in the art, these are merely examples and intended to be non-limiting as other methods, techniques, and types of techniques are contemplated and possible.

In one embodiment performance of a function may include providing an alert to one or more users or remote systems. For example, the system may transmit a notification to a user indicating that the water quality at a particular location is poor or outside acceptable ranges. As another example, the system may provide a pop-up message indicating that the water quality at a particular location is acceptable. Provision of an alert or notification may include transmitting the alert or notification to a third party. For example, the system may transmit an alert to the utility that provides the fluid, for example, a water utility. As another example, the system may transmit an alert to a supplier of the fluid. The alert may provide an indication of the water quality. Additionally, the alert may provide a requested action. For example, the system may send an alert to a maintenance team or response team to perform maintenance on the point-of-use delivery system.

In one embodiment performance of a function may include providing instructions to the water fountain to disable the fountain or shut-off the fountain. Accordingly, the point-of-use system may include a shut-off mechanism, for example, a mechanical shut-off mechanism (e.g., valve, flapper, pump shut-off, diverter, magnetic shut-off, etc.), an electrical shut-off (e.g., a circuit breaker, fuse, etc.), a software shut-off (e.g., a processor that prevents dispensing of the fluid, a code shut-down, etc.) or a combination thereof. In the event that the water quality is determined to be poor, for example, the correlation of the pH measurement, which may include temperature correction, and conductivity/ORP measurement to water quality has indicated that the water quality is poor, the processing system may provide instructions to the shut-off mechanism or a controller that controls the shut-off mechanism to activate the shut-off mechanism so that the point-of-use system cannot dispense the fluid.

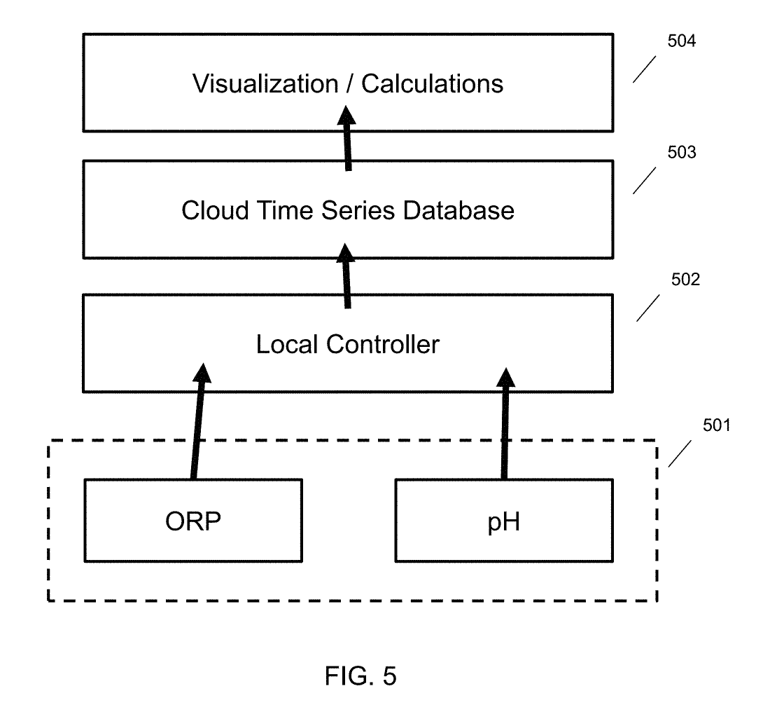

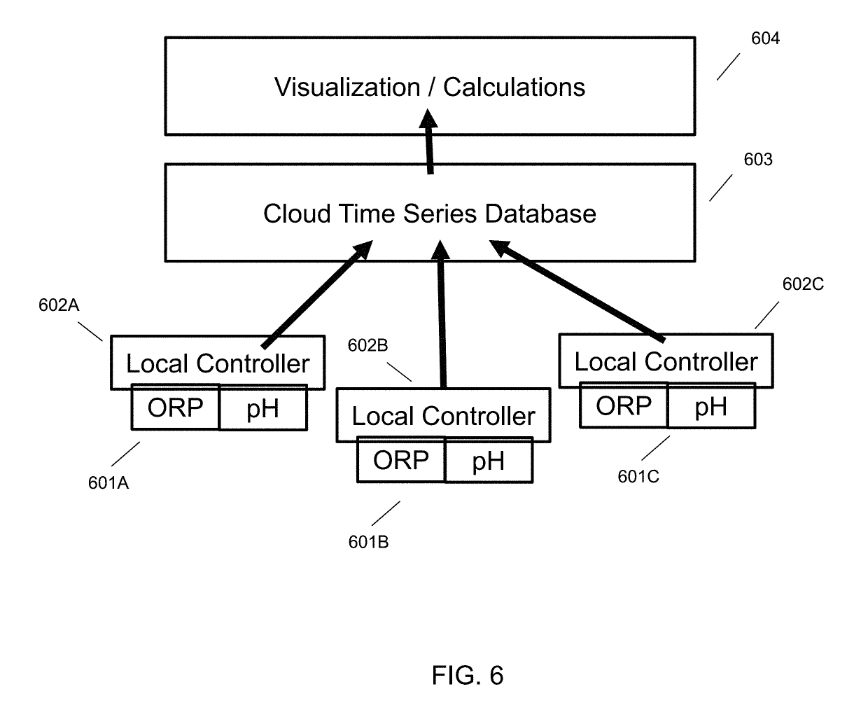

Not only can the systems and techniques as described herein be used to determine the water quality of a single point, for example as shown in FIG. 5, but they may also be used to determine locations of failures or points of failure. The processing/analysis system may receive information from many different point-of-use systems, for example, as shown in FIG. 6, which may include all the water fountains located in a particular building, all the houses in a particular region, or the like. As shown in FIGS. 5 and 6, one device (as shown in FIG. 5 at 501) or multiple devices (as shown in FIG. 6 at 601A-601C) may provide information to a local controller 502 and 602 which may be located at the system or at a remote location operatively coupled to the system. The local controller 502 and 602A-602C may then provide this information or a derivative of this information to the cloud database 503 and 603, which then may process and analyze the information to complete calculations and provide visualizations at 504 and 604.

Using the measurement information from multiple locations, the system may make comparisons between other similar objects or point-of-use delivery systems and determine a point or cause of failure or contamination. In one embodiment the system may overlay the calculated values or quality values (e.g., good, bad, poor, acceptable, etc.) over a map. Using this map, a user or the processing system may identify whether point-of-use systems having poor quality have a common characteristic. For example, the system may determine if the point-of-use systems are all connected to the same supply line, are all in the same region, are all of the same type (e.g., all water fountains, all sinks, etc.), or the like.

If the system can identify a common characteristic, the system may be able to determine a point of failure or a possible cause of contamination. For example, if all the point-of-use systems are connected to the same supply line, the system may determine that the supply line may be providing the contamination. As another example, the system may identify that one hundred houses are connected to the same main trunk line. The system may then identify that the houses are grouped into groups of twenty-five houses with each group having a common branch line. The system may determine that if all one hundred houses have poor quality water, then the contamination may be caused by the trunk line. Whereas if only one or two groupings of the houses have poor quality water, then the contamination may be caused by one or more of the branch lines. As a contrasting scenario, if the point-of-use systems that have poor quality water are sporadic and do not have a common characteristic, the system may determine or infer that the cause of contamination may be unique to the point-of-use system. For example, the contamination may be caused by the piping in the house, rather than the utility supply. Once the system determines or identifies a possible cause of failure or contamination, the system may notify one or more users of the possible cause of failure or contamination.

The various embodiments described herein thus represent a technical improvement to conventional systems for identifying a fluid quality. The systems and methods as described herein provide technique for using low cost measurement sensors that can be added at point-of-use locations for a minimal cost. Using the measurements captured by these sensors the system may calculate a water quality measurement to determine if the water it outside a desired threshold range, thereby providing reassurance to consumers without a significant cost to utilities or other water providers. Accordingly, the systems and methods as described herein provide a low cost alternative to conventional water quality measurement techniques.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and “non-transitory” includes all media except signal media.

Program code embodied on a storage medium may be transmitted using any appropriate medium, including but not limited to wireless, wireline, optical fiber cable, RF, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A point-of-use fluid delivery system, comprising:
a vessel containing a fluid;
at least one sensor capturing data related to a first measurement comprising a pH measurement;
at least one other sensor capturing data related to a second measurement selected from the group consisting of: an ORP measurement and a conductivity measurement of the fluid;
a processor; and
a memory device operatively coupled to the processor and that stores instructions executable by the processor to transmit the data related to the pH and/or temperature measurement of the fluid and the data related to the conductivity and/or ORP measurement of the fluid to a remote system.

2. The point-of-use fluid delivery system of claim 1, further comprising a fluid sample chamber.

3. The point-of-use fluid delivery system of claim 2, wherein a fluid sample is drawn into the fluid sample chamber from the vessel.

4. The point-of-use fluid delivery system of claim 3, wherein the at least one sensor and the at least one other sensor are located at the fluid sample chamber.

5. The point-of-use fluid delivery system of claim 1, further comprising a shut-off mechanism.

6. The point-of-use fluid delivery system of claim 5, wherein the shut-off mechanism is activated in response to instructions from the remote system to shut-off the fluid delivery system.

7. The point-of-use fluid delivery system of claim 6, wherein the instructions to shut-off the fluid delivery system are provided in response to an identification of a quality of the fluid being less than a predetermined level as calculated based upon the data related to the first measurement of the fluid and the data related to the second measurement of the fluid.

8. The point-of-use fluid delivery system of claim 1, wherein the vessel comprises at least one of a tube or a pipe.

9. The point-of-use fluid delivery system of claim 1, wherein the point-of-use fluid delivery system comprises one of the devices selected from the group consisting of: a water fountain, a home fluid delivery system, and a business fluid delivery system.

10. The point-of-use fluid delivery system of claim 1, wherein the fluid comprises water.

11. The point-of-use fluid delivery system of claim 1, further comprising a data transmission device for transmitting the data related to the first measurement of the fluid and the data related to the second measurement of the fluid to a remote system

12. A system, comprising:
a processor;
a memory device operatively coupled to the processor, wherein the memory device comprises instructions executable by the processor to:
capture, using at least one sensor of a fluid delivery system, data related to a first measurement comprising a pH measurement of a fluid sample;
capture, using at least one other sensor of the fluid delivery system, data related to a second measurement selected from the group consisting of: an ORP measurement and a conductivity measurement of the fluid sample; and
transmit the first measurement data and the second measurement data to a remote system for identifying a quality of the fluid sample determined by correlating the first measurement data and the second measurement data to quality information.

13. The system of claim 12, further comprising instructions to shut-off the fluid delivery system in response to receiving a shut-off signal from the remote system.

14. The system of claim 13, wherein the shut-off signal is generated by the remote system in response to identifying a quality of the fluid sample is outside a predetermined threshold.

15. The system of claim 12, further comprising instructions to draw the fluid sample into a fluid sample chamber of the fluid delivery system.

16. The system of claim 12, wherein the instructions to capture the first measurement data and the second measurement data comprises instructions to capture the first measurement data and the second measurement data at predetermined intervals.

17. A water fountain system, comprising:
tubing containing water;
a water sample chamber connected to the tubing, wherein the water sample chamber draws water into the water sample chamber;
at least one sensor, connected to the water sample chamber, capturing data related to a first measurement comprising a pH measurement of the fluid;
at least one other sensor, connected to the water sample chamber, capturing data related to a second measurement selected from the group consisting of: an ORP measurement and a conductivity measurement of the fluid;
a data transmission device;
a processor; and
a memory device operatively coupled to the processor and that stores instructions executable by the processor to transmit, using the data transmission device, the data related to the first measurement of the fluid and the data related to the second measurement of the fluid to a remote system.

18. The water fountain system of claim 17, further comprising a shut-off mechanism.

19. The point-of-use fluid delivery system of claim 18, wherein the shut-off mechanism is activated in response to instructions from the remote system to shut-off the fluid delivery system.

20. The point-of-use fluid delivery system of claim 19, wherein the instructions to shut-off the fluid delivery system are provided in response to an identification of a quality of the fluid being less than a predetermined level as calculated based upon the data related to the first measurement of the fluid and the data related to the second measurement of the fluid.
